(19) United States
(12) Patent Application Publication
(10) Pub. No.: US 2022/0257585 A1
(43) Pub. Date: Aug. 18, 2022

(54) HYALURONIC ACID-BASED OPHTHALMIC DRUG DELIVERY SYSTEM, AND METHOD FOR PRODUCING SAME

(71) Applicant: DONGGUK UNIVERSITY INDUSTRY-ACADEMIC COOPERATION FOUNDATION, Seoul (KR)

(72) Inventors: Choul Yong PARK, Goyang-si, Gyeonggi-do (KR); Mi Young JUNG, Seoul (KR); Joo Hee PARK, Seoul (KR)

(21) Appl. No.: 17/618,611

(22) PCT Filed: Jun. 9, 2020
(86) PCT No.: PCT/KR2020/007455

§ 371 Date: Dec. 13, 2021

(30) Foreign Application Priority Data
Jun. 14, 2019 (KR) 10-2019-0070444

(51) Int. Cl. A61K 31/4709; A61K 9/00; A61K 47/36; A61K 47/08

(52) U.S. Cl. CPC A61K 31/4709; A61K 47/08; A61K 47/36; A61K 9/0051

(57) ABSTRACT

Disclosed is an ophthalmic drug delivery system intended for insertion into an eye, the ophthalmic drug delivery system containing: a carrier comprising crosslinked hyaluronic acid; and a drug loaded in the carrier. Also disclosed is a method for producing an ophthalmic drug delivery system, the method comprising steps of: preparing a first solution containing hyaluronic acid and water; preparing a second solution containing 1,4-butanediol diglycidyl ether (BDDE) and water; sterilizing the first solution and the second solution separately; preparing a third solution by mixing the sterilized first solution and second solution together; and polymerizing the hyaluronic acid by incubating the third solution.

### TECHNICAL FIELD

The present invention relates to an ophthalmic drug delivery system based on hyaluronic acid and a method for producing the same.

### BACKGROUND ART

With an aging population, the number of glaucoma or cataract patients is increasing, and eye surgery therefor is also increasing. After eye surgery, antibacterial agents in the form of eye drops are administered to the eyes to prevent bacterial infection, and it is important to administer the antibacterial agent at a consistent and accurate dose for 7 to 14 days immediately after surgery. The antibacterial agent is administered at a total dose for effectively preventing bacterial infection, and the dose and frequency of administration thereof should be determined in consideration of the in vivo half-life thereof.

Moxifloxacin (MXF) is an antibacterial agent mainly used in ophthalmic surgery, and is administered as an eye drop to prevent bacterial infection after glaucoma or cataract surgery. The total dose of moxifloxacin per week is about 3.5 mg, the content of moxifloxacin in an eye drop is about 0.5 wt %, and the in vivo half-life of moxifloxacin is about 12.7 hours. Considering these facts, moxifloxacin is recommended to be instilled at least twice a day in an amount of about 50 μL per time. However, since the field of vision is limited after surgery, it is difficult to instill moxifloxacin by itself. In addition, in many cases, a correct amount of moxifloxacin is not administered because the eye drop flows down or the timing of instillation is not observed. In addition, since subjects who have undergone ophthalmic surgery are often elderly, it is often difficult to accurately instill the eye drop on their own.

Hyaluronic acid (HA) is a linear polysaccharide composed of repeating disaccharide units of beta-D-N-acetylglucosamine and beta-D-glucuronic acid. Hyaluronic acid, a compound of extracellular tissue found in vivo, is known to be biocompatible and biodegradable in vivo. However, hyaluronic acid is rapidly degraded in vivo and thus is difficult to use for sustained-release drug release.

In order to solve the inconvenience of instilling moxifloxacin, studies have been conducted on drug delivery systems loaded with moxifloxacin and on drug carriers with chitosan and dextran (Kaskoos, R.A. Investigation of moxifloxacin loaded chitosan-dextran nanoparticles for topical instillation into eye: In-vitro and ex-vivo evaluation. Int. J. Pharm. Investig. 2014, 4, 164-173). However, an ophthalmic drug delivery system based on hyaluronic acid has not yet been known.

In addition, a sterilization process is required in the production of a hyaluronic acid-based drug delivery system intended for insertion into an eye. However, when the sterilization process is performed, a problem arises in that the time during which the polymerized state of hyaluronic acid is retained is rapidly shortened, making it difficult to sustainedly release the drug loaded therein for a certain period of time.

### DISCLOSURE

### Technical Problem

According to one embodiment, there is provided an ophthalmic drug delivery system based on hyaluronic acid, which is capable of stably releasing an antibiotic by maintaining the physical properties thereof for a certain period of time even after a sterilization process, and a method for producing the same.

### Technical Solution

One aspect provides an ophthalmic drug delivery system intended for insertion into an eye, the ophthalmic drug delivery system containing: a carrier comprising cross-linked hyaluronic acid; and a drug loaded in the carrier.

The ophthalmic drug delivery system has no toxicity when inserted into the eye, and may replace the administration of an eye drop after surgery by sustainedly releasing the loaded drug while being slowly degraded in the eye for a certain period of time.

The drug may be a steroid, a nitric oxide releasing agent, or an antibacterial agent. The antibacterial agent may be a fluoroquinolone antibiotic. The fluoroquinolone antibiotic has a structure in which fluorine is added at the C-6 position of quinoline, and is characterized by exhibiting a broad spectrum of antibacterial activity against both gram-positive and gram-negative bacteria.

Examples of the fluoroquinolone antibiotic include ciprofloxacin, ofloxacin, levofloxacin, moxifloxacin, and the like. The nitric oxide releasing agent may be a substance having a diazeniumdiolate (NONOate) group produced by reacting nitric oxide with an amine group. The nitric oxide-releasing agent may be polyethylenimine (PEI) treated with nitric oxide, and more specifically, branched PEI treated with nitric oxide. In one embodiment, the drug may be moxifloxacin.

The IUPAC name of moxifloxacin is 1-cyclopropyl-7-[(1S,6S)-2,8-diazabicyclo [4.3.0]non-8-yl]-6-fluoro-8-methoxy-4-oxo-quinoline-3-carboxylic acid. Moxifloxacin is used for the treatment of bacterial conjunctivitis, keratitis, etc. caused by bacterial species such as Staphylococcus or Streptococcus, or for aseptic therapy before and after ophthalmic surgery.

In one embodiment, the content ratio between the hyaluronic acid and the drug may be, on a mass basis, 1:1 to 300:1, 2:1 to 300:1, 5:1 to 300:1, 10:1 to 300:1, 20:1 to 300:1, 30:1 to 300:1, 60:1 to 300:1, 90:1 to 300:1, 120:1 to 300:1, 150:1 to 300:1, 180:1 to 300:1, 210:1 to 300:1, 240:1 to 300:1, 270:1 to 300:1, 1:1 to 150:1, 2:1 to 150:1, 5:1 to 150:1, 10:1 to 150:1, 20:1 to 150:1, 30:1 to 150:1, 60:1 to 150:1, 90:1 to 150:1, 120:1 to 150:1, 1:1 to 60:1, 2:1 to 60:1, 5:1 to 60:1, 10:1 to 60:1, 20:1 to 60:1, or 30:1 to 60:1, preferably 30:1.

In one embodiment, the content ratio between the hyaluronic acid and moxifloxacin may be, on a mass basis, 1:1 to 300:1, 2:1 to 300:1, 5:1 to 300:1, 10:1 to 300:1, 20:1 to 300:1, 30:1 to 300:1, 60:1 to 300:1, 90:1 to 300:1, 120:1 to 300:1, 150:1 to 300:1, 180:1 to 300:1, 210:1 to 300:1, 240:1 to 300:1, 270:1 to 300:1, 1:1 to 150:1, 2:1 to 150:1, 5:1 to 150:1, 10:1 to 150:1, 20:1 to 150:1, 30:1 to 150:1, 60:1 to 150:1, 90:1 to 150:1, 120:1 to 150:1, 1:1 to 60:1, 2:1 to 60:1, 5:1 to 60:1, 10:1 to 60:1, 20:1 to 60:1, or 30:1 to 60:1, preferably 30:1. The ratio of 30:1 is a value determined in consideration of the concentration of the antibiotic generally required against bacterial infection of the eye when the antibiotic contained in the drug delivery system is diluted with the anterior chamber water in the eye. The content ratio may be appropriately adjusted in consideration of symptoms, the risk of bacterial infection, etc. The present inventors have found that, when a drug delivery system produced by incorporating 0.1 to 3 wt % of moxifloxacin into 3 wt % of hyaluronic acid is injected into the anterior chamber of the eye, degradation thereof may be delayed for at least one week in an environment in which it hydrated by anterior chamber water, and side effects thereof may be minimized.

In one embodiment, the crosslinking may be achieved by an epoxide crosslinking agent. The epoxide crosslinking agent may be at least one selected from among 1,4-butanediol diglycidyl ether (BDDE), ethylene glycol diglycidyl ether (EGDGE), 1,6-hexanediol diglycidyl ether, propylene glycol diglycidyl ether, poly(propylene glycol)diglycidyl ether, poly(tetramethylene glycol)diglycidyl ether, neopentyl glycol diglycidyl ether, polyglycerol polyglycidyl ether, diglycerol polyglycidyl ether, glycerol polyglycidyl ether, trimethylpropane polyglycidyl ether, 1,2-(bis(2,3-epoxypropoxy)ethylene, pentaerythritol polyglycidyl ether, and sorbitol polyglycidyl ether.

In particular, the drug delivery system may be obtained by crosslinking using 1,4-butanediol diglycidyl ether (BDDE) as a crosslinking agent. When a hyaluronic acid complex is produced using BDDE as a crosslinking agent, the concentration of residual BDDE may be easily controlled below a dangerous level, thereby minimizing side effects that may occur upon insertion into an eye.

In one embodiment, the ophthalmic drug delivery system may be obtained by mixing the hyaluronic acid and the epoxide crosslinking agent together at a mass ratio of 150:1 to 600:1, 250:1 to 600:1, 300:1 to 600:1, 150:1 to 500:1, 250:1 to 500:1, 300:1 to 500:1, 150:1 to 400:1, 250:1 to 400:1, 300:1 to 400:1, or 300:1, followed by crosslinking.

In one embodiment, the ophthalmic drug delivery system may be obtained by mixing the hyaluronic acid and BDDE together at a mass ratio of 150:1 to 600:1, 250:1 to 600:1, 300:1 to 600:1, 150:1 to 500:1, 250:1 to 500:1, 300:1 to 500:1, 150:1 to 400:1, 250:1 to 400:1, 300:1 to 400:1, or 300:1, followed by crosslinking. If the content ratio of BDDE is increased, the degree of crosslinking of hyaluronic acid may increase and the degradation of the drug delivery system in the eye may be delayed so that the drug release period may increase, but the residual amount of BDDE may increase, leading to increased side effects. On the other hand, if the content ratio of BDDE is lowered, it is difficult to secure a sufficient drug release period. The appropriate drug release period of the drug delivery system inserted into the eye may be at least 5 days, preferably at least 7 days, and at most 10 days, at most 11 days, at most 12 days, or at most 14 days. According to an embodiment of the present invention, it was confirmed that, when the content ratio between hyaluronic acid and BDDE was set to 300:1 on a mass ratio basis, degradation of the hyaluronic acid complex in the eye could be appropriately delayed, so that the hyaluronic acid complex could be retained for at least 7 days, which is the period required for antibiotic instillation after surgery, and could be retained for up to 10 days, and the risk of side effects thereof due to toxicity was low.

In one embodiment, the intraocular position into which the drug delivery system is to be inserted may be the anterior chamber or the vitreous cavity. The anterior chamber and the vitreous cavity are medical terms meaning positions within the eye, and the meanings thereof are well known to those skilled in the art to which the present invention pertains, and may be understood with reference to FIG. 1. The present inventors inserted the drug delivery system according to one embodiment into the anterior chamber of a rabbit eye and checked the retention period thereof. As a result, the present inventors confirmed that the drug delivery system could be retained in the anterior chamber for at least 7 days, which is the period required for antibiotic instillation after surgery, and showed no toxicity.

In one embodiment, the insertion may be insertion after ophthalmic surgery, and specifically, it may be insertion before suturing after incision in the ophthalmic surgery process. The surgery is a surgery which requires the administration of an antibiotic to prevent bacterial infection after surgery, and is not limited to a specific surgery. The ophthalmic drug delivery system according to one embodiment may replace administration of an eye drop by being inserted into an eye before suturing in a surgical process, and thus may be advantageously used for recovery after surgery.

In one embodiment, the ophthalmic drug delivery system may be produced by crosslinking a solution containing hyaluronic acid, BDDE, and moxifloxacin. The solution may contain hyaluronic acid in an amount of 2.5 to 3.5 wt %, 2.8 to 3.2 wt %, or 3 wt %, and contain BDDE in an amount of 0.005 to 0.015 wt %, 0.008 to 0.012 wt %, or 0.009 to 0.011 wt %, and contain the drug in an amount of 0.01 to 0.15 wt %, 0.03 to 0.15 wt %, 0.05 to 0.15 wt %, 0.08 to 0.15 wt %, 0.01 to 0.1 wt %, 0.03 to 0.1 wt %, 0.05 to 0.1 wt %, 0.08 to 0.1 wt %, 0.01 to 0.05 wt %, or 0.03 to 0.05 wt %. According to this embodiment, the drug delivery system produced by crosslinking a solution containing 3 wt % of hyaluronic acid, 0.01 wt % of BDDE and 0.1 wt % of the drug may release the drug for at least 7 days when inserted into the anterior chamber of an eye, and side effects thereof may be minimized.

The crosslinking may comprise first crosslinking and second crosslinking which is performed after injecting a solution, subjected to the first crosslinking, into a molding mold. The first crosslinking reaction may be performed for 12 to 30 hours, 15 to 30 hours, 16 to 30 hours, 17 to 30 hours, 18 to 30 hours, 19 to 30 hours, 12 to 27 hours, 15 to 27 hours, 16 to 27 hours, 17 to 27 hours, 18 to 27 hours, 19 to 27 hours, 12 to 24 hours, 15 to 24 hours, 16 to 24 hours, 17 to 24 hours, 18 to 24 hours, 19 to 24 hours, 12 to 21 hours, 15 to 21 hours, 16 to 21 hours, 17 to 21 hours, 18 to 21 hours, or 19 to 21 hours, and may preferably be performed for 19 hours.

The ophthalmic drug delivery system may be stored in a dry state, and may be hydrated for use prior to administration.

The dried ophthalmic drug delivery system may be processed to have various sizes, lengths and shapes, which are not particularly limited. Preferably, the dried ophthalmic drug delivery system may be processed to have an appropriate size and shape in consideration of the frequency of administration and the total content of moxifloxacin to be released during the duration thereof in an eye.

The size of the ophthalmic drug delivery system may be 0.1 μm to 5 mm in diameter or long axis.

The shape of the ophthalmic drug delivery system may be spherical, cylindrical, rectangular, film, hemispherical, amorphous, lenticular, nanocarrier, granular, or fine particle shape.

The method for administration of the ophthalmic drug delivery system is not particularly limited as long as it is a method for administration into an eye. For example, the dried drug delivery system may be hydrated and inserted with forceps, and if necessary, it may be administered by injection.

The administration timing of the ophthalmic drug delivery system may be before suturing after surgery, and the number of administrations thereof may be once. The total content of moxifloxacin in the drug delivery system used for single administration may vary depending on the patient's condition, weight, and recovery period, and may be, for example, 3 to 4 mg, or 3.5 mg.

The single dose of the ophthalmic drug delivery system may be, on a dry weight basis, 2.5 to 3.0 mg, 2.6 to 3.0 mg, 2.7 to 3.0 mg, 2.5 to 2.9 mg, 2.6 to 2.9 mg, 2.7 to 2.9 mg, 2.5 to 2.8 mg, 2.6 to 2.8 mg, 2.7 to 2.8 mg, 2.5 to 2.7 mg, or 2.6 to 2.7 mg. The present inventors have found that, when administered at the above-described single dose, the ophthalmic drug delivery system may release the drug while being retained in the eye for the period required for antibiotic administration after surgery. The ophthalmic drug delivery system may be administered after hydration, and the mass thereof when hydrated may be 55 to 90 mg, 60 to 90 mg, 65 to 90 mg, 55 to 85 mg, 60 to 85 mg, 65 to 85 mg, 55 to 80 mg, 60 to 80 mg, or 65 to 80 mg.

Another aspect provides a method for producing an ophthalmic drug delivery system, the method comprising steps of: mixing hyaluronic acid, BDDE and moxifloxacin together in a basic aqueous solution; and crosslinking the mixture.

The description of the hyaluronic acid, BDDE and moxifloxacin and the mixing ratio therebetween are the same as those described above.

The step of crosslinking may comprise a first crosslinking step and a second crosslinking step of injecting and molding the mixture into a mold.

The production method may further comprise a step of drying the drug delivery system.

The production method may comprise preparing a hyaluronic acid solution as a first solution and a BDDE solution as a second solution, sterilizing the solutions separately, and mixing the solutions together, followed by the first crosslinking step.

Still another aspect provides a method for producing an ophthalmic drug delivery system, the method comprising steps of: preparing a first solution containing hyaluronic acid and water; preparing a second solution containing BDDE (1,4-butanediol diglycidyl ether) and water; sterilizing the first solution and the second solution separately; preparing a third solution by mixing the sterilized first solution and second solution together; and polymerizing the hyaluronic acid by incubating the third solution.

The present inventors have found that, when a drug delivery system is produced using hyaluronic acid and BDDE without performing a separate sterilization process, bacteria may proliferate in the produced drug delivery system after hydration. To overcome this problem, the present inventors have conducted repeated studies, and as a result, have established a process for producing a hyaluronic acid complex, which is capable of preventing bacterial proliferation after hydration by comprising a sterilization process, while retaining the shape thereof for at least one week even after hydration by achieving a high degree of crosslinking of hyaluronic acid.

The ophthalmic drug delivery system may load a drug, and may replace the administration of an eye drop after surgery by sustainedly releasing the loaded drug while being slowly degraded in vivo for a certain period of time.

The drug may be a steroid, a nitric oxide releasing agent, or an antibacterial agent. The antibacterial agent may be a fluoroquinolone antibiotic. The fluoroquinolone antibiotic has a structure in which fluorine is added at the C-6 position of quinoline, and is characterized by exhibiting a broad spectrum of antibacterial activity against both gram-positive and gram-negative bacteria. Examples of the fluoroquinolone antibiotic include ciprofloxacin, ofloxacin, levofloxacin, moxifloxacin, and the like. The nitric oxide-releasing agent may be a substance having a diazeniumdiolate (NONOate) group produced by reacting nitric oxide with an amine group. The nitric oxide-releasing agent may be polyethylenimine (PEI) treated with nitric oxide, and more specifically, branched PEI treated with nitric oxide.

In one embodiment, the first solution may contain the hyaluronic acid in an amount of 4 to 8 wt %, 5 to 8 wt %, 6 to 8 wt %, 4 to 7 wt %, 5 to 7 wt %, or 6 to 7 wt %, preferably 6 wt %.

In one embodiment, the second solution may contain the BDDE in an amount of 0.015 to 0.025 wt %, 0.018 to 0.025 wt %, 0.019 to 0.025 wt %, 0.015 to 0.022 wt %, 0.018 to 0.022 wt %, 0.019 to 0.022 wt %, 0.015 to 0.021 wt %, 0.018 to 0.021 wt %, or 0.019 to 0.021 wt %, preferably 0.02 wt %. For example, if the amount of the second solution is the same as that of the first solution and the first solution contains 6 wt % of hyaluronic acid, the second solution may contain 0.02 wt % of BDDE. According to the contents of hyaluronic acid and BDDE, it is possible to increase the period during which the shape of the hyaluronic acid complex is retained in vivo, and to minimize toxicity caused by BDDE. According to the contents of hyaluronic acid and BDDE, the shape of the hyaluronic acid complex may be retained in vivo for the period required for antibiotic instillation after surgery, and the period of the retention may be about 5 to 10 days, 6 to 10 days, 7 to 10 days, 8 to 10 days, 5 to 9 days, 6 to 9 days, 7 to 9 days, 8 to 9 days, 5 to 8 days, 6 to 8 days, 7 to 8 days, 5 to 7 days, or 6 to 7 days, preferably 7 days.

In one embodiment, the content ratio between hyaluronic acid in the first solution and BDDE in the second solution may be 400:1, 390:1, 380:1, 370:1, 360:1, 350:1, 340:1, 330:1, 320:1, 310:1, 300:1, 290:1, 280:1, 270:1, 260:1, or 250:1, preferably 300:1, on a weight basis.

In one embodiment, the first solution, the second solution, or the mixture of the solutions may contain moxifloxacin in an amount equal to 1/600 to 1/10 of the content of hyaluronic acid.

In one embodiment, the content of hyaluronic acid in the third solution may be 2.5 to 3.5 wt %, or 2.8 to 3.2 wt %, and the content of BDDE in the third solution may be 0.008 to 0.012 wt %, or 0.009 to 0.011 wt %. Preferably, the third solution may contain 3 wt % of hyaluronic acid and 0.01 wt % of BDDE.

In one embodiment, the sterilizing step may be sterilization with high pressure steam, an autoclave, ethylene oxide (EO) gas, gamma rays, electron beams, or ultraviolet rays. Preferably, it may be autoclave sterilization. The present inventors have found that, when a hyaluronic acid polymer is produced by performing a sterilization process after the crosslinking reaction of hyaluronic acid and BDDE, the time during which the shape of the polymer after hydration is retained is excessively shortened, making it difficult to use the polymer as a drug delivery system. In addition, the present inventors have found that, when the first crosslinking reaction is performed after sterilization of each of hyaluronic acid and BDDE, the time during which the shape of the polymer after hydration is retained is at least 7 days, and the shape of the polymer may be retained for a period during which an antibiotic-containing eye drop is administered after surgery.

In one embodiment, the polymerizing step may be performed for 12 to 30 hours, 15 to 30 hours, 16 to 30 hours, 17 to 30 hours, 18 to 30 hours, 19 to 30 hours, 12 to 27 hours, 15 to 27 hours, 16 to 27 hours, 17 to 27 hours, 18 to 27 hours, 19 to 27 hours, 12 to 24 hours, 15 to 24 hours, 16 to 24 hours, 17 to 24 hours, 18 to 24 hours, 19 to 24 hours, 12 to 21 hours, 15 to 21 hours, 16 to 21 hours, 17 to 21 hours, 18 to 21 hours, 19 to 21 hours, preferably, 19 hours.

In one embodiment, the polymerizing step may comprise injecting and incubating the mixture of the solutions into a molding mold.

In one embodiment, the step of preparing the third solution may be performed in an aseptic facility. For example, it may be performed in a clean bench.

The ophthalmic drug delivery system contains an antibiotic loaded therein, and may be used for administration of the antibiotic after eye surgery. The ophthalmic drug delivery system produced by the above-described production method may remain in vivo for a period almost identical to the period required for antibiotic instillation after surgery, and used as a substitute for antibiotic instillation after surgery by being inserted into an eye during eye surgery.

### Advantageous Effects

Since the ophthalmic drug delivery system according to one embodiment has excellent biocompatibility and biodegradability, side effects thereof, such as an increase in intraocular pressure or an inflammatory reaction, after insertion into an eye, may be minimized.

The ophthalmic drug delivery system loaded with a drug according to one embodiment may release the loaded drug for the period during which infection prevention after surgery is required, while degradation thereof in the eye is delayed for at least one week.

The ophthalmic drug delivery system loaded with a drug according to one embodiment may replace administration of an eye drop by being inserted into an eye during eye surgery and releasing the drug in the eye.

The ophthalmic drug delivery system loaded with a drug according to one embodiment may reduce the amount of antibiotic used compared to administration of a conventional eye drop.

### BRIEF DESCRIPTION OF DRAWINGS

FIG. 1 shows the anatomical structure of an eye.

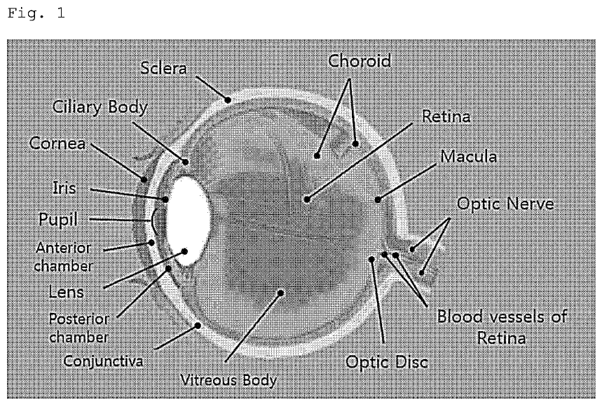

FIG. 2 shows an HA complex film in dry state, produced according to an example of the present invention.

FIG. 3 shows the thickness of the HA complex film in dry state, produced according to an example of the present invention.

FIG. 4 shows the results obtained by swelling the HA complex film, produced according to an example of the present invention, through absorption of saline, placing the film on the dorsal surface of a hand, and confirming that the shape of the film would be retained.

FIG. 5 shows the results obtained by cutting the HA complex film in dry state, produced according to an example of the present invention, into various forms, and confirming that the shape of the film after hydration would be retained.

FIG. 6 shows the results obtained by hydrating and then drying the HA complex film, produced according to an example of the present invention, on a PVDF film, and examining the shape retention ability of the film for up to 3 weeks.

FIG. 7 shows a mixture solution prepared by mixing moxifloxacin with hyaluronic acid and BDDE.

FIG. 8 shows the results of examining the physical properties of an HA complex film in dry state, produced using the mixture solution shown in FIG. 7.

FIG. 9 shows the results of observing the shape of the moxifloxacin-containing HA complex film shown in FIG. 8 at one week after hydration.

FIG. 10 shows the results of an MIC test for a drug delivery system containing moxifloxacin.

FIG. 11 shows the results of evaluating the cytotoxicity of an HA complex film, produced according to an example of the present invention, to human corneal epithelial cells.

FIG. 12 shows the results obtained by inserting the HA complex film, produced according to an example of the present invention, into the eye of an experimental animal, and evaluating the biocompatibility and biodegradability of the film.

FIG. 13 shows the results obtained by inserting the HA complex film, produced according to an example of the present invention, into the wounded cornea of an experimental animal, and confirming biocompatibility and an increase in recovery rate.

FIG. 14 shows the results obtained by hydrating the HA complex film, produced by first crosslinking and sterilization, and confirming whether the shape of the film would be retained.

FIG. 15 shows the results of hydrating HA complex films. Specifically, FIG. 15A shows an HA complex film produced by first crosslinking and then sterilization, and FIG. 15B shows an HA complex film produced by sterilizing a hyaluronic acid solution and a BDDE solution separately and performing first crosslinking.

FIG. 16 shows SEM photographs of HA complex films produced by different methods. Specifically, FIG. 16A shows an HA complex film produced without sterilization, FIG. 16B shows an HA complex film produced by a first crosslinking reaction and then sterilization with an autoclave, and FIG. 16C shows an HA complex film produced by sterilizing a hyaluronic acid solution and a BDDE solution separately and performing first crosslinking. The scale bar in each photograph is 500 μm.

FIG. 17 shows the results of measuring the pore size based on the SEM photographs of FIG. 16. “CTL” is for FIG. 16A, “autoclaved” is for FIG. 16B, and “separately autoclaved” is for FIG. 16C.

### MODE FOR INVENTION

Hereinafter, one or more specific embodiments will be described in more detail with reference to examples. However, these examples serve to illustrate one or more embodiments, and the scope of the present invention is not limited to these examples.

### Example 1: Production of Crosslinked Hyaluronic Acid

A solution was prepared by mixing hyaluronic acid and BDDE with water at pH 8.0. Based on the total weight of the solution, 3% of hyaluronic acid and 0.01% of BDDE were mixed. The total volume of the prepared solution was 30 to 40 ml.

The prepared solution was subjected to a first crosslinking reaction by mixing the solution by 360° rotation using bio RS-24 mini rotator (Biosan) at room temperature for 19 hours or more. The solution subjected to the first crosslinking reaction was placed in a culture plate lid for molding. A second crosslinking reaction was performed by incubating the culture plate lid containing the solution overnight in a shaker (CR300 rocker, FINEPCR).

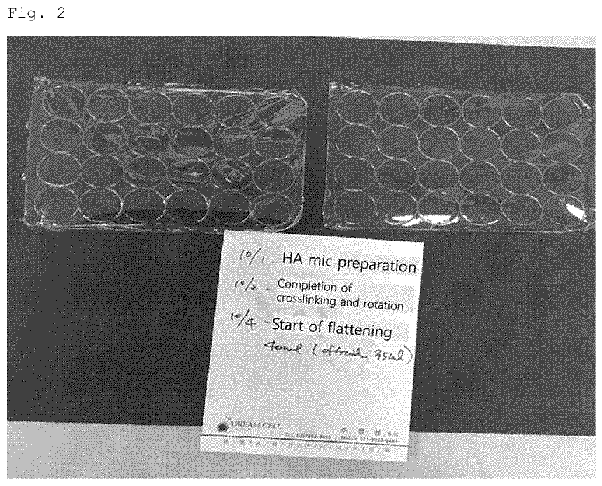

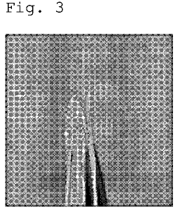

After completion of the second crosslinking reaction, the flat hyaluronic acid film was dried on a table for 3 days or more. The dried hyaluronic acid film was cut (trimmed) by punching into a circular flat shape having a diameter of about 5 mm, and used in the experiment (see FIG. 2). The cut hyaluronic acid film was named HA complex film. The thickness of the HA complex film was about 100 to 125 μm (see FIG. 3).

### Example 2: Evaluation of Physical Properties of Flat HA Complex Film

2-1. Evaluation of Physical Properties Through Attachment to Dorsal Surface of Hand

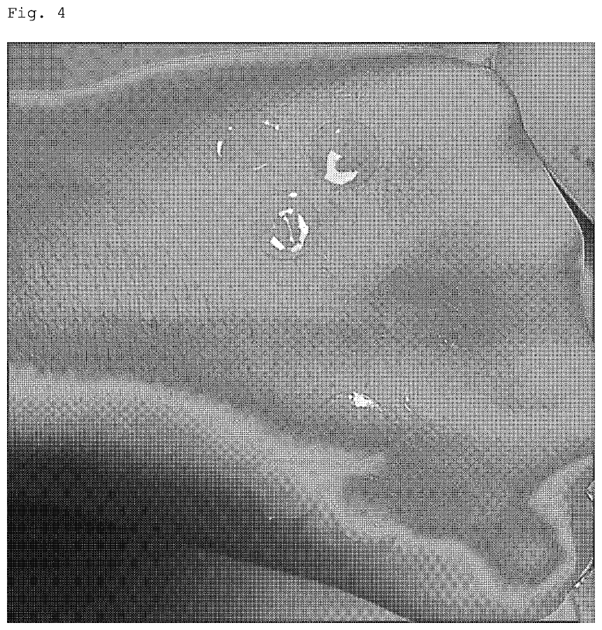

The HA complex film produced in Example 1 was swollen through absorption of normal saline for 15 minutes, and attached to the dorsal surface of a hand (see FIG. 4). As a result, it could be confirmed that the HA complex film absorbed water to have a flat shape swollen to a diameter of about 5 mm to about 8 mm, and had excellent adhesion to a curved surface.

From the results of Example 2-1, it could be confirmed that the hyaluronic acid film produced according to the composition of Example 1 had excellent adhesion to biological tissues when hydrated, and could retain its polymerized state and shape even when re-dried after hydration.

2-2. Evaluation of Physical Properties of HA Complex Films Having Various Shapes

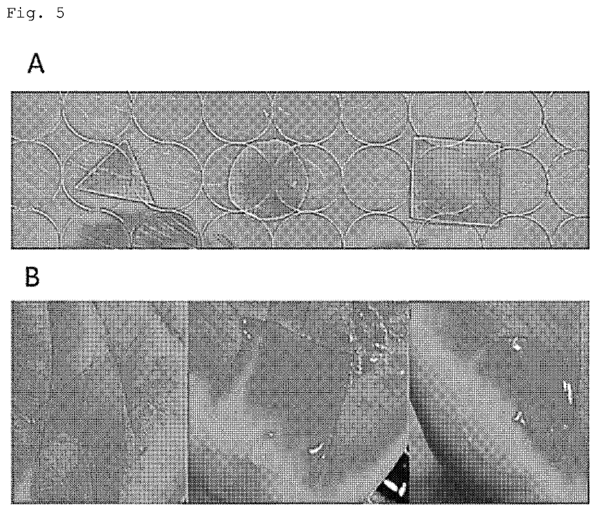

As a result of cutting the HA complex film into an angular shape (FIG. 5A) rather than a circular shape and swelling the cut film in the same manner, it was confirmed that the cut film could be in close contact with the skin and could retain its shape even after hydration (FIG. 5B).

2-3. Evaluation of Physical Properties after Placement on PVDF Film

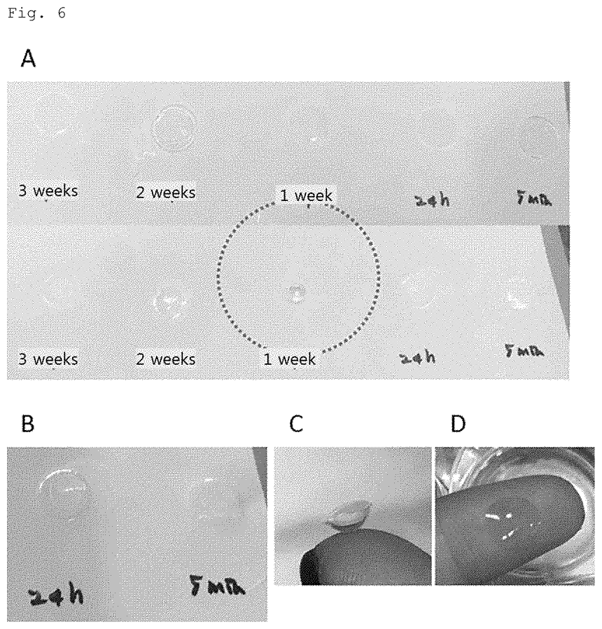

The HA complex film was allowed to absorb normal saline for different periods of time, and the change in the shape thereof upon drying was observed. FIG. 6A shows the results obtained by placing the HA complex film on a PVDF film, allowing the HA complex film to absorb normal saline, and then drying the HA complex film for 1 hour. In FIG. 6A, each of 3 weeks, 2 weeks, 1 week, 24 h, 5 min means the time during which the HA complex film was swollen by absorbing normal saline. Referring to FIG. 6B, the dried HA complex film was in close contact with the PVDF film and was cracked. This is believed to be due to drying of the film on the PVDF film. However, referring to the circle portion indicated by a dotted line in FIG. 6A and to FIG. 6C, it was confirmed that, even when the HA complex film was allowed to absorb normal saline and dried for 1 week, it was completely dried in the form of a contact lens, and when it was allowed again to absorb normal saline, it was swollen and restored into a flat shape (FIG. 6D). Therefore, it was confirmed that the HA complex film produced according to the above-described composition could retain its shape in the intraocular environment for one week or more.

### Example 3: Production of HA Complex Film Loaded with Moxifloxacin and Evaluation of Physical Properties Thereof

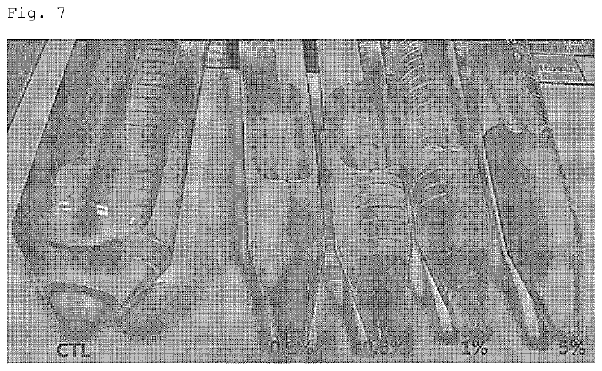

An HA complex film was produced in the same manner as in Example 1, except that moxifloxacin was further mixed in the step of mixing hyaluronic acid and BDDE. Referring to FIG. 7, it could be confirmed that the yellow color of the mixture solution became darker as the mass % of the mixed moxifloxacin increased.

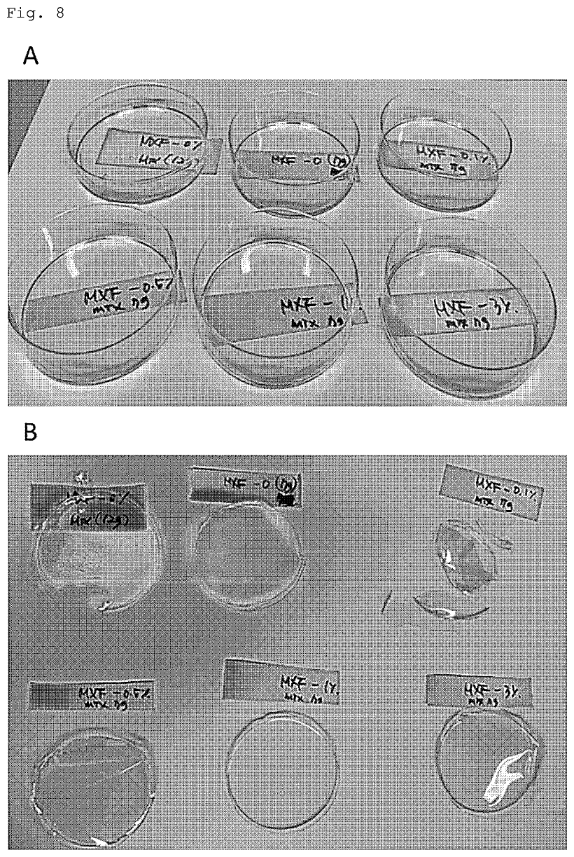

A mixture solution containing hyaluronic acid, BDDE and moxifloxacin was placed in a Petri dish having a diameter of about 5 cm, flattened, subjected to a sufficient crosslinking reaction, and then dried for 4 days (see FIG. 8A). As a result of separating the dried moxifloxacin-loaded HA complex film from the Petri dish, it could be confirmed that the shape of the HA complex film could be retained even when it contained up to 3 wt % of moxifloxacin (see FIG. 8B).

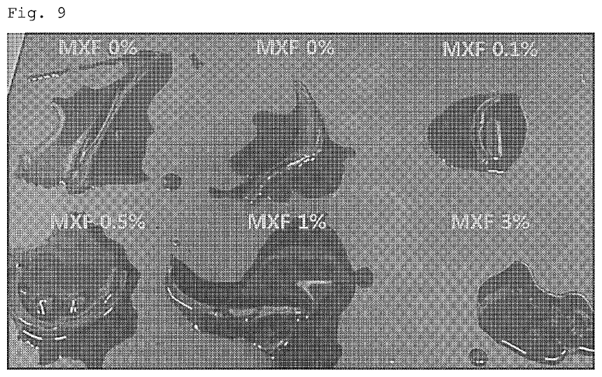

In order to measure the intraocular duration of the HA complex film containing moxifloxacin, the HA complex film was hydrated and the degree of degradation thereof was observed for one week. Referring to FIG. 9, it was evaluated that the degradation of the HA complex film containing 3 wt % of moxifloxacin progressed slightly, but the difference in the content of moxifloxacin did not lead to a significant difference in the degradation rate.

### Example 5: Evaluation of Bacterial Inhibition Ability of HA Complex Film Containing Moxifloxacin

HA complex films having different contents of moxifloxacin were produced, and the minimum concentration that inhibits bacterial growth was measured by performing a minimum inhibitory concentration (MIC) test.

Each of the hyaluronic acid complex films containing 0, 0.1, 0.5, 1 and 3 wt % of moxifloxacin, respectively, was added to a medium inoculated with Pseudomonas aeruginosa, followed by shaking incubation at 37° C. After 24 hours, the absorbance (optical density) at a wavelength of 600 nm was measured with a spectrophotometer, and then the increase or decrease in the number of bacteria was expressed as a percentage relative to the control taken as 100%.

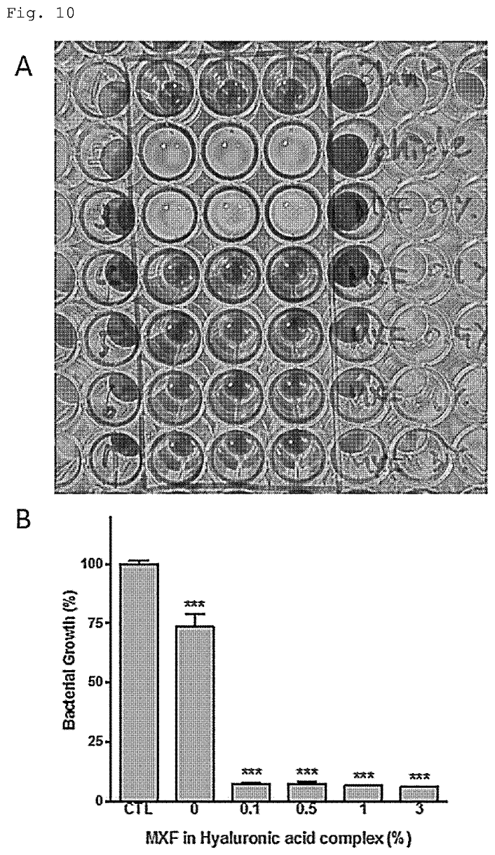

Referring to the color change shown in FIG. 10A and the absorbance measurement results shown in FIG. 10B, it could be confirmed that, in the case of the HA complex film containing no moxifloxacin, the color changed due to the proliferation of Pseudomonas aeruginosa, but the hyaluronic acid complex films produced to contain 0.1 wt % or more of moxifloxacin effectively inhibited the proliferation of Pseudomonas aeruginosa.

### Example 6: Toxicity Test (CCK Test) for HA Complex

Film for Human Corneal Epithelial Cells

Primary human corneal epithelial (HCEpi) cells were cultured for 4 passages in a 24-well plate. The number of the cultured cells was about 3×10⁴ cells/well. The hyaluronic acid complex was added to the cell culture, and the cells were cultured for 24 to 72 hours, and then whether the HA complex would be toxic to the cells was evaluated by a CCK test.

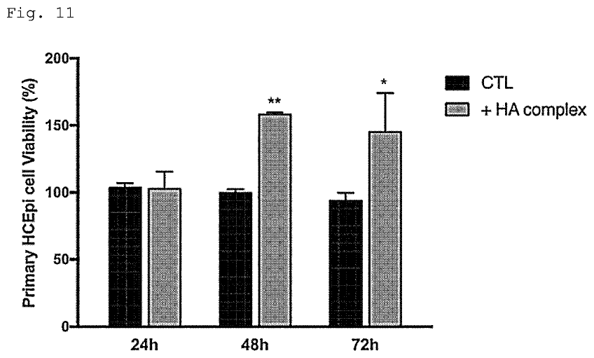

FIG. 11 shows CCK data obtained by comparing the cell viability after treatment with the hyaluronic acid complex. According to the experimental results, it was confirmed that the cell viability was higher in the group treated with the hyaluronic acid complex than in the untreated group. Therefore, it was confirmed that the hyaluronic acid complex of the present invention has no toxicity problem when inserted into an eye and rather helps cell survival.

### Example 7: Evaluation of Biocompatibility, Biodegradability and Duration of HA Complex Film

The HA complex film produced in Example 1 was inserted into the anterior chamber of a rabbit eye, and the biocompatibility and biodegradability thereof were tested.

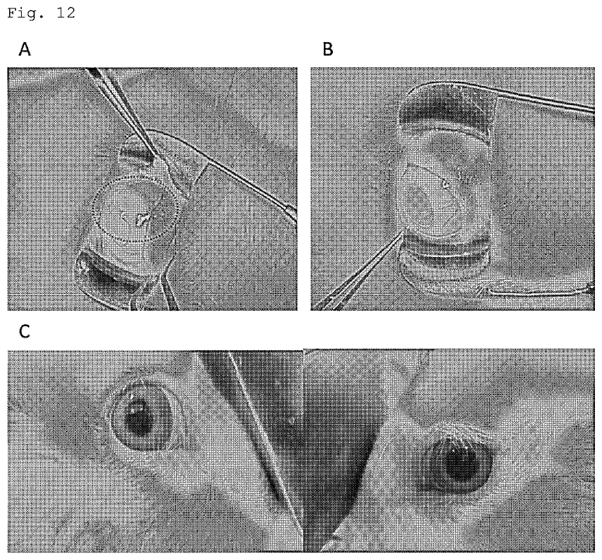

The average dry weight of the hyaluronic acid complex inserted into the anterior chamber of the rabbit eye was 2.7 mg, and the weight thereof when hydrated by adding an average of 200 μL of water or normal saline was 65 to 80 mg. FIG. 12A shows a state in which the conjunctiva and cornea were incised and the HA complex film was inserted into the anterior chamber. In FIG. 12A, the inserted HA complex film is indicated by a dotted line. FIG. 12B shows a state in which the cornea and conjunctiva were sutured.

FIG. 12C shows the result of observing the eye of the rabbit at one week after cornea suturing. It was confirmed that the HA complex film was not observed due to biodegradation 7 days after insertion into the eye, suggesting that the HA complex film can last for at least 7 days in an in vivo environment. In addition, in terms of biocompatibility, there was some swelling after inserting the HA complex into the eye, but this swelling is thought to be due to the aftereffects of the surgery, and other side effects due to the HA complex film were not observed, suggesting that the HA complex film has excellent biocompatibility. Based on these results, it is expected that, when the HA complex film loaded with a drug is used in the same manner as described above, the loaded drug may be released sustainedly at the insertion site for one week, and the HA complex film may be used without any special side effects.

In addition, for a total of 5 rabbits (2 rabbits for a control and 3 rabbits for an experimental group), each eye was damaged by scratching the cornea in a circular shape of 1 cm in diameter, and then the size of the wound site was checked at different time points by staining with fluorescein. In the experimental group, the corneal wound was covered with the HA complex film, obtained by punching with a diameter of 5 mm and hydration.

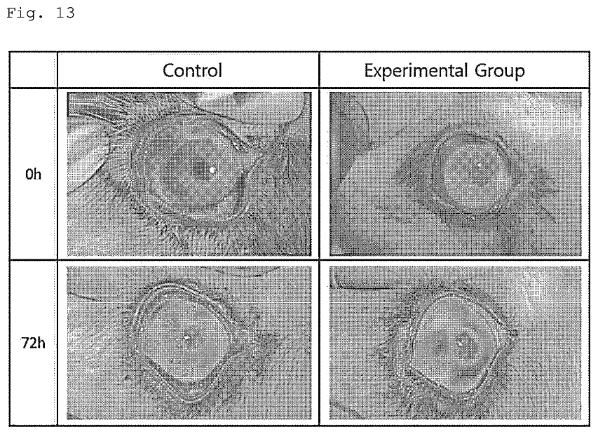

Referring to FIG. 13, it could be confirmed that, 72 hours after corneal damage, the stained area in the control group partially decreased, whereas the stained area in the experimental group completely disappeared, suggesting that the HA complex film promoted recovery from corneal damage.

### Example 8: Establishment of Sterilization Process in

Production of HA Complex Film [00114]8-1. Carrying out of Sterilization after First

Crosslinking

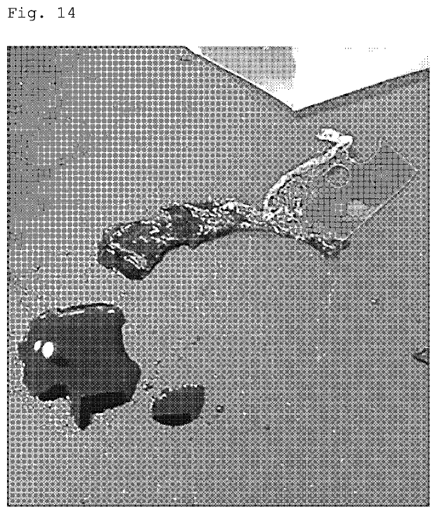

After the processes up to the first crosslinking reaction by the rotator in the method of Example 1 were performed in the same manner as in Example 1, autoclave sterilization was performed at 121° C. for 20 minutes. The sterilized first crosslinking reaction solution was placed in a culture plate lid for molding. A second crosslinking reaction was performed by incubating the culture plate lid containing the solution overnight in a shaker (CR300 rocker, FINEPCR). All processes after sterilization were performed on a clean bench. After the second crosslinking reaction, the flat HA complex film was dried on a table for at least 3 days. It was confirmed that, when the dried HA complex film was hydrated, it was degraded within a few minutes and could not retain its shape (see FIG. 14).

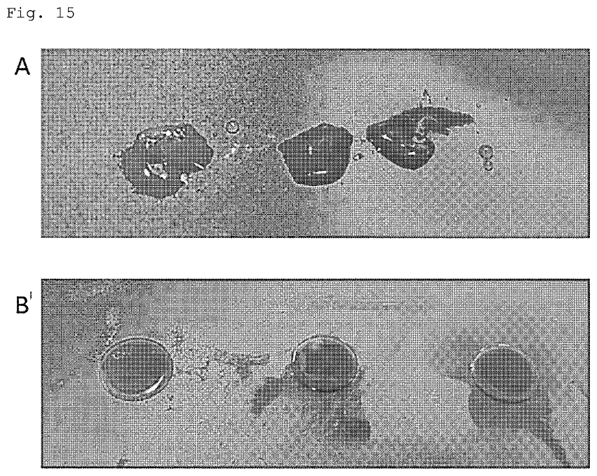

HA complex films were prepared in the same manner as descried above, except that the sterilization process was performed by each of E0 gas sterilization (55° C. for 1 to 2 hours) at a lower temperature than autoclave sterilization, gamma sterilization (at each of 25 kgy and 40 kgy), and electron beam sterilization (at each of 25 kgy and 40 kgy). However, it was confirmed that, when the HA complex films sterilized by the above-described various methods were hydrated, they were also degraded within minutes and could not retain their shape. In addition, it was confirmed that, when the HA complex film produced using the UV sterilization process as the sterilization process was hydrated, the shape thereof disappeared within 44 to 51 hours after hydration (see FIG. 15A).

Thereby, it was confirmed that, when sterilization is performed after the first crosslinking reaction, sustained drug release in the eye is difficult. [00118]8-2. Sterilization of Each of Hyaluronic Acid Solution and Crosslinking Agent Solution [00119]20 ml of a solution of 6 wt % hyaluronic acid (pH 8.0) as a first solution and 20 ml of a solution of 0.02 wt % BDDE (pH 8.0) as a second solution were prepared separately. Each of the first solution and the second solution was autoclave-sterilized under the same conditions as in Example 6-1, and a third solution was prepared by mixing the sterilized first solution and second solution together, and then subjected to a first crosslinking reaction using a rotator for 19 hours. The third solution subjected to the first crosslinking reaction was placed in a culture plate lid. A second crosslinking reaction was performed by incubating the culture plate lid containing the solution overnight in a shaker (CR300 rocker, FINEPCR). All processes after sterilization were performed on a clean bench. After the second crosslinking reaction, the flat HA complex film was dried on a table for 3 days or more.

As a result of hydrating the HA complex film produced using each sterilized solution and observing the film while continuously supplying water thereto, it was confirmed that the shape thereof was retained for 96 hours or more (see FIG. 15B).

### Example 9: Results of Scanning Electron Microscope Analysis of HA Complex Film Produced in Example 8-2

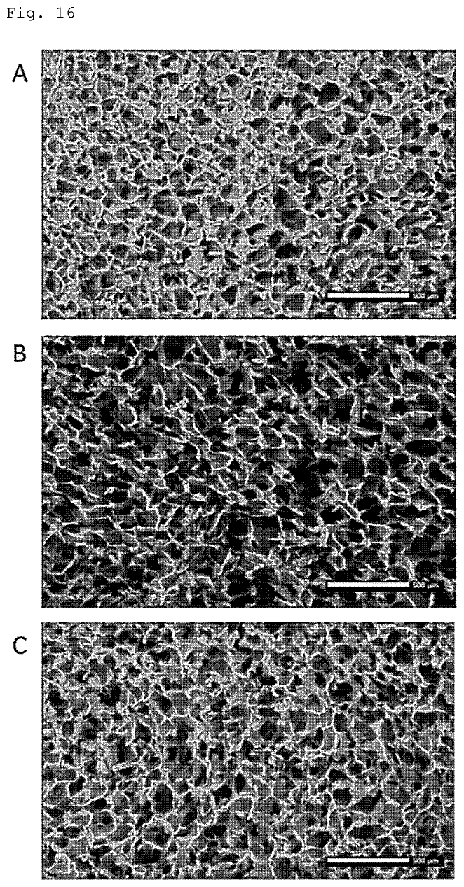

FIG. 16 shows the results of scanning electron microscope (SEM) observation of the HA complex film produced according to the method of Example 1 without a sterilization process (FIG. 16A), the HA complex film autoclave-sterilized after the first crosslinking reaction according to the method of Example 6-1 (FIG. 16B), and the HA complex film produced by sterilization of each of the hyaluronic acid solution and the BDDE solution and the first crosslinking reaction according to the method of Example 6-2 (FIG. 16C). The scanning electron microscopy was performed under conditions of an SED of 15.0 kV and a WD of 9.7 mm, and the scale bar represents a length of 500 μm.

Referring to FIGS. 16A, 16B and 16C, it was confirmed that the shape and size of the crosslinked pores were different between the films. The pores shown in FIGS. 16A and 16C were similar in shape and size. However, referring to FIG. 16B, it could be confirmed that a large number of relatively larger pores were observed, and the spacing between the pores was not dense, suggesting that crosslinking was not achieved properly.

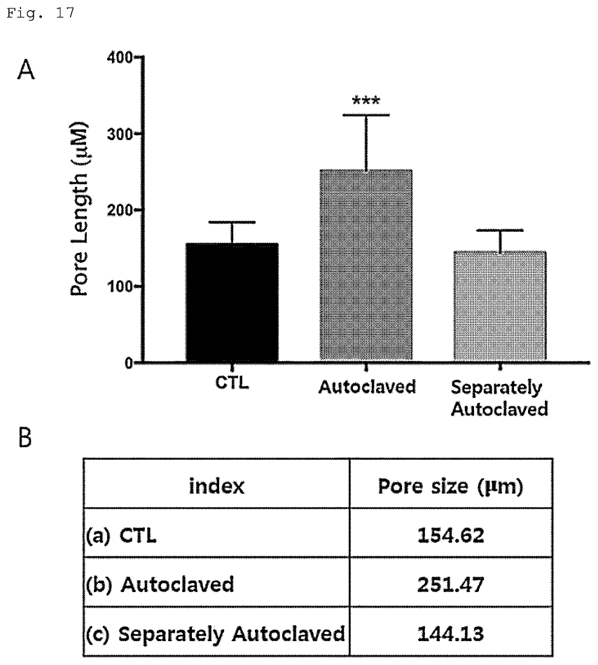

The results of measuring the average size of the pores are shown in FIG. 17. In FIGS. 17A and 17B, CTL is the result corresponding to FIG. 16A, “autoclaved” is the result corresponding to FIG. 16B, and “separately autoclaved” is the result corresponding to FIG. 16C. It was confirmed that the average pore size in FIG. 16A was 154.62 μm (CTL in FIG. 17), the average pore size in FIG. 16B was 251.47 μm (“autoclaved” in FIG. 17), and the average pore size in FIG. 16C was 144.13 μm (“separately autoclaved” in FIG. 17). It could be confirmed that, when autoclave sterilization was performed after the first crosslinking reaction, the pore size significantly increased, suggesting that the autoclave sterilization process damaged the crosslinking during the process of producing the hyaluronic acid complex. In addition, it could be confirmed that, when the hyaluronic acid solution and the BDDE solution were sterilized separately and subjected to the first crosslinking reaction, the crosslinking of the hyaluronic acid was not damaged because the chemical crosslinking by BDDE was performed after the sterilization process.

In addition, a conventional hyaluronic acid complex produced without sterilization treatment often had a problem of bacterial proliferation, but when each solution was sterilized and subjected to the first crosslinking reaction, no bacterial proliferation occurred in the hyaluronic acid complex while the complex retained its shape for one week or more when hydrated as described above.

Therefore, it was confirmed that the HA complex produced by performing the first crosslinking reaction after sterilizing the hyaluronic acid solution and the crosslinking agent solution separately can maintain its shape for a longer period in the intraocular environment, and thus it may be advantageously used for the production of a drug delivery system capable of sustained drug release.

1. An ophthalmic drug delivery system intended for insertion into an eye, the ophthalmic drug delivery system containing:
a carrier comprising crosslinked hyaluronic acid; and
a drug loaded in the carrier.

2. The ophthalmic drug delivery system of claim 1, wherein the drug is a steroid, a nitric oxide releasing agent, or an antibacterial agent.

3. The ophthalmic drug delivery system of claim 2, wherein the antibacterial agent is a fluoroquinolone antibiotic.

4. The ophthalmic drug delivery system of claim 3, wherein the fluoroquinolone antibiotic is moxifloxacin.

5. The ophthalmic drug delivery system of claim 1, wherein a content ratio between the hyaluronic acid and the drug is 30:1 to 300:1 on a mass basis.

6. The ophthalmic drug delivery system of claim 4, wherein a content ratio between the hyaluronic acid and the moxifloxacin is 30:1 to 300:1 on a mass basis.

7. The ophthalmic drug delivery system of claim 1, wherein the crosslinking is achieved by an epoxide crosslinking agent.

8. The ophthalmic drug delivery system of claim 7, which is obtained by mixing the hyaluronic acid and the epoxide crosslinking agent together at a mass ratio of 30:1 to 300:1, followed by crosslinking.

9. The ophthalmic drug delivery system of claim 7, wherein the epoxide crosslinking agent is 1,4-butanediol diglycidyl ether (BDDE).

10. The ophthalmic drug delivery system of claim 9, which is obtained by mixing the hyaluronic acid and BDDE together at a mass ratio of 150:1 to 600:1, followed by crosslinking.

11. The ophthalmic drug delivery system of claim 1, which releases the drug in the eye for 7 to 10 days.

12. The ophthalmic drug delivery system of claim 1, wherein the eye is an anterior chamber or a vitreous body.

13. The ophthalmic drug delivery system of claim 1, wherein the insertion is insertion after ophthalmic surgery.

14. The ophthalmic drug delivery system of claim 1, which is produced by mixing 2.5 to 3.5 wt % of hyaluronic acid, 0.005 to 0.015 wt % of BDDE and 0.05 to 0.15 wt % of the drug together and crosslinking the hyaluronic acid.

15. The ophthalmic drug delivery system of claim 1, which is administered at a single dose of 2.5 to 3.0 mg as a dry weight basis.

16. A method for producing an ophthalmic drug delivery system, the method comprising steps of:
preparing a first solution containing hyaluronic acid and water;
preparing a second solution containing 1,4-butanediol diglycidyl ether (BDDE) and water;
sterilizing the first solution and the second solution separately;
preparing a third solution by mixing the sterilized first solution and second solution together; and
polymerizing the hyaluronic acid by incubating the third solution.

17. The method of claim 16, wherein the first solution contains 4 to 8 wt % of the hyaluronic acid.

18. The method of claim 16, wherein the second solution contains 0.015 to 0.025 wt % of the BDDE.

19. The method of claim 16, wherein a content ratio between the hyaluronic acid in the first solution and the BDDE in the second solution is 300:1 on a weight basis.

20. The method of claim 16, wherein the third solution has a hyaluronic acid content of 2.5 to 3.5 wt % and a BDDE content of 0.008 to 0.012 wt %.

21. The method of claim 16, wherein the sterilization is performed with an autoclave.

22. The method of claim 16, wherein the step of polymerizing is performed for 17 to 21 hours.

23. The method of claim 16, wherein the step of polymerizing comprises injecting and incubating the mixture of the solutions into a molding mold.
